(19) United States
(12) Patent Application Publication
(10) Pub. No.: US 2020/0316349 A1
(43) Pub. Date: Oct. 8, 2020

(54) Micro-Camera Guide Wire

(71) Applicant: Fraser M. Smith, Salt Lake City, UT (US)

(72) Inventor: Fraser M. Smith, Salt Lake City, UT (US)

(21) Appl. No.: 16/374,680

(22) Filed: Apr. 3, 2019

(51) Int. Cl. A61M 25/09; A61B 1/05

(52) U.S. Cl. CPC A61M 25/09033; A61M 2025/0166; A61B 1/051

(57) ABSTRACT

A method of advancing a guidewire having an imaging device disposed thereon within a cavity of a body includes placing a guidewire into a cavity of a body, said guidewire having an image sensor disposed about a guide member on a distal end of the guidewire and a plurality of power lines operatively coupled to the guide member and the image sensor. At least two power lines of the plurality of power lines are disposed on opposing sides of the guide member and extend from the distal end of the guidewire to a proximal end of the guidewire. As the guidewire is advanced through the body cavity, the distal end of the guidewire is positioned by pulling one of the at least two power lines while maintaining the position of the other of the at least two power lines.

### FIELD OF THE TECHNOLOGY

The present technology relates to improved devices, methods, and systems for medical devices. More particularly, the present technology relates to a micro-camera disposed on the distal end of a guide wire.

### BACKGROUND OF THE TECHNOLOGY AND RELATED ART

Catheter guide wires have been used for many years to “lead” or “guide” catheters to desired target locations in the human body's vasculature. A conventional guide wire ranges from about 135 centimeters to 195 centimeters in length, and is made from two primary pieces—a stainless steel core wire, and a platinum alloy coil spring. The core wire is tapered on the distal end to increase its flexibility. The coil spring is typically soldered to the core wire at a point where the inside diameter of the coil spring matches the outside diameter of the core wire. Platinum is selected for the coil spring because it provides radiopacity for X-ray viewing during navigation of the guide wire in the body, and it is biocompatible. The coil spring also provides softness for the tip of the guide wire to reduce the likelihood of a puncture of the anatomy.

Navigation through the anatomy can be achieved by viewing the guide wire in the body using X-ray fluoroscopy. The guide wire is inserted into a catheter so the guide wire protrudes out the end, and then the wire and catheter are inserted into a vessel or duct and moved therethrough until the guide wire tip reaches a desired vessel or duct branch. The proximal end of the guide wire is then rotated or torqued to point the curved tip into the desired branch and then advanced further. The catheter is advanced over the guide wire to follow or track the wire to the desired location, and provide additional support for the wire. Once the catheter is in place, the guide wire may be withdrawn, depending upon the therapy to be performed. Oftentimes, such as in the case of balloon angioplasty, the guide wire is left in place during the procedure and will be used to exchange catheters.

As the guide wire is advanced into the anatomy, internal resistance from the typically numerous turns, and surface contact, decreases the ability to advance the guide wire further. In addition, the ability to properly navigate internal vasculature is increased by technical difficulties associated with external detection of the location of the guide wire within the vasculature. This, in turn, may lead to a more difficult and prolonged procedure, or, more seriously, failure to access the desired anatomy and thus a failed procedure. A guide wire with improved steering controls and internal viewing capabilities will help overcome problems created by the internal resistance and challenge in viewing the tortuous cavities of a patient.

### SUMMARY OF THE INVENTION

In light of the problems and deficiencies inherent in the prior art, the present invention seeks to overcome these by providing methods, devices, and systems for an elongate medical device configured for placement into a cavity of a body.

The present disclosure sets forth a method of advancing a guidewire having an imaging device disposed thereon within a cavity of a body, comprising placing a guidewire into a cavity of a body, said guidewire comprising an image sensor disposed about a guide member on a distal end of the guidewire and a plurality of power lines operatively coupled to the guide member and the image sensor, wherein at least two power lines of the plurality of power lines are disposed on opposing sides of the guide member and extend from the distal end of the guidewire to a proximal end of the guidewire; advancing the guidewire through the body cavity while viewing an internal area of the body cavity with a display device coupled to the image sensor to a target location within the body; positioning the distal end of the guidewire within the body cavity applying a force to one of the at least two power lines while maintaining the position of the other of the at least two power lines; placing a catheter about an exterior of the guidewire and advancing the catheter through the body cavity; and removing the guidewire from the body cavity while leaving the catheter in the body cavity.

The present disclosure also sets forth a method of advancing a guidewire having an imaging device disposed thereon within a cavity of a body, comprising placing a guidewire into a cavity of a body, said guidewire comprising (i) an image sensor disposed about a guide member on a distal end of the guidewire, and (ii) a single power line operatively coupled to the guide member and the image sensor, wherein the single power line extends from a bottom of the guide member up through a first aperture in the guide member across the guide member and down through a second aperture in the guide member, the first and second apertures being disposed on laterally opposing sides of the guide member, creating opposing cables; advancing the guidewire through the body cavity; and positioning the distal end of the guidewire within the body cavity by pulling on one of the opposing cables of the single power line while maintaining the position of the opposing cable of the single power line.

The present disclosure further sets forth a guidewire configured for placement into a cavity of a body, comprising an elongate body member having a proximal end and a distal end, the elongate body member comprising a light source and an image sensor disposed about the distal end of the body; a guide member disposed about the distal end of the elongate body, wherein the image sensor is disposed about a top surface of the guide member; and a plurality of tensioning members disposed about opposing lateral sides of the guide member, wherein each tensioning member comprises a single cable of tensioning material forming a loop about the guide member, said loop extending from below the guide member, up through an aperture within the guide member and back below a bottom of the guide member.

The present disclosure still further sets forth a guidewire configured for placement into a cavity of a body, comprising an elongate body member having a proximal end and a distal end, the elongate body member comprising a light source and an image sensor disposed about the distal end of the body; a guide member disposed about the distal end of the elongate body, wherein the image sensor is disposed about the guide member; and a single cable tensioning member extending (i) from a bottom of the guide member up through an aperture in the guide member, (ii) laterally across the guide member, and (iii) back below a bottom of the guide member.

### BRIEF DESCRIPTION OF THE DRAWINGS

The present technology will become more fully apparent from the following description and appended claims, taken in conjunction with the accompanying drawings. Understanding that these drawings merely depict exemplary aspects of the present technology they are, therefore, not to be considered limiting of its scope. It will be readily appreciated that the components of the present technology, as generally described and illustrated in the figures herein, could be arranged and designed in a wide variety of different configurations. Nonetheless, the technology will be described and explained with additional specificity and detail through the use of the accompanying drawings in which:

FIG. 1 is a schematic illustration of one aspect of a medical imaging system in accordance with certain principles of the technology;

FIG. 2 is a cross-sectional side view of the distal end of a guidewire in accordance with one aspect of the technology;

FIG. 3 is a cross-sectional side view of the distal end of a guidewire in accordance with one aspect of the technology;

FIG. 4 is a perspective view of the distal end of a guidewire in accordance with one aspect of the technology;

FIG. 5 is a perspective view of the distal end of a guidewire in accordance with one aspect of the technology;

FIG. 6 is a perspective view of a guide member disposed about the distal end of a guidewire in accordance with one aspect of the technology;

FIG. 7 is a perspective view of a guide member disposed about the distal end of a guidewire in accordance with one aspect of the technology;

FIG. 8 is a perspective view of a guide member having a core disposed about the distal end of a guidewire in accordance with one aspect of the technology;

FIG. 9 is a top view of a guide member in accordance with one aspect of the technology;

FIG. 10 is a top view of a guide member in accordance with one aspect of the technology;

FIG. 11 is a cross-sectional side view of the distal end of a guidewire in accordance with one aspect of the technology;

FIG. 12 is a top view of a guide member in accordance with one aspect of the technology;

FIG. 13 is a cross-sectional side view of a distal end of a portion of a guide member in accordance with one aspect of the technology; and

FIG. 14 is a cross-sectional side view of a distal end of a portion of a guide member in accordance with one aspect of the technology.

### DETAILED DESCRIPTION OF EXEMPLARY ASPECTS OF THE TECHNOLOGY

The following detailed description of exemplary aspects of the technology makes reference to the accompanying drawings, which form a part hereof and in which are shown, by way of illustration, exemplary aspects in which the technology may be practiced. While these exemplary aspects are described in sufficient detail to enable those skilled in the art to practice the technology, it should be understood that other aspects may be realized and that various changes to the technology may be made without departing from the spirit and scope of the present technology. Thus, the following more detailed description of the aspects of the present technology is not intended to limit the scope of the technology, as claimed, but is presented for purposes of illustration only and not limitation to describe the features and characteristics of the present technology, to set forth the best mode of operation of the technology, and to sufficiently enable one skilled in the art to practice the technology. Accordingly, the scope of the present technology is to be defined solely by the appended claims. The following detailed description and exemplary aspects of the technology will be best understood by reference to the accompanying drawings, wherein the elements and features of the technology are designated by numerals throughout.

As used in this specification and the appended claims, the singular forms “a,” “an” and “the” include plural referents unless the context clearly dictates otherwise. Thus, for example, reference to “a layer” includes a plurality of such layers.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.”

A “SSID,” “solid state imaging device,” “SSID chip,” or “solid state imaging chip” in the exemplary embodiments generally comprises an imaging array or pixel array for gathering image data. In one embodiment, the SSID can comprise a silicon or silicon-like substrate or amorphous silicon thin film transistors (TFT) having features typically manufactured therein. Features can include the imaging array, conductive pads, metal traces, circuitry, etc. Other integrated circuit components can also be present for desired applications. However, it is not required that all of these components be present, as long as there is a means of gathering visual or photon data, and a means of sending that data to provide a visual image or image reconstruction.

The term “umbilical” can include the collection of utilities that operate the SSID or the micro-camera as a whole. Typically, an umbilical includes a conductive line, such as electrical wire(s) or other conductors, for providing power, ground, clock signal, and output signal with respect to the SSID, though not all of these are strictly required. For example, ground can be provided by another means than through an electrical wire, e.g., to a camera housing. The umbilical can also include other utilities such as a light source, temperature sensors, force sensors, fluid irrigation or aspiration members, pressure sensors, fiber optics, microforceps, material retrieval tools, drug delivery devices, radiation emitting devices, laser diodes, electric cauterizers, and electric stimulators, for example. Other utilities will also be apparent to those skilled in the art and are thus comprehended by this disclosure.

“GRIN lens” or “GRadient refractive INdex lens” refers to a specialized lens that has a refractive index that is varied radially from a center optical axis to the outer diameter of the lens. In one embodiment, such a lens can be configured in a cylindrical shape, with the optical axis extending from a first flat end to a second flat end. Thus, because of the differing refractive index in a radial direction from the optical axis, a lens of this shape can simulate the effects of a. more traditionally shaped lens.

The directional terms “proximal” and “distal” are used herein to refer to opposite locations on a medical device. The proximal end of a device is defined as the end closest to the practitioner when the device is being used or manipulated by a practitioner. The distal end is the end opposite the proximal end, along the longitudinal direction of the device, or the end furthest from the practitioner. It is understood that, as used in the art, these terms may have different meanings with regard to devices deployed within the body of a patient (i.e., the “proximal” end may refer to the end closest to the head or heart of the patient depending on the application). For consistency, as used herein, the ends labeled “proximal” and “distal” prior to deployment remain the same regardless of whether the device is disposed within a patient.

“Lens” as used herein means a transmissive optical device which affects the focusing of a light beam through refraction. The lens may comprise a single piece of material or several materials disposed along a common axis.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

An initial overview of technology is provided below and specific technology is then described in further detail. This initial summary is intended to aid readers in understanding the technology more quickly, but is not intended to identify key or essential features of the technology, nor is it intended to limit the scope of the claimed subject matter.

Aspects of the current technology are intended to provide improved systems, devices, and methods of placing a micro-camera disposed about a guidewire within a cavity or vessel of a patient. The guidewire comprises an image sensor disposed about a guide member on a distal end of the guidewire and a plurality of power lines operatively coupled to the image sensor. At least two power lines of the plurality of power lines are disposed on opposing sides of the guide member and extend from the distal end of the guidewire to a proximal end of the guidewire. As the guidewire is advanced through the vasculature (or other body cavity) of the patient, the distal end of the guidewire is steered into different vessels (or cavities) by pulling on one of the opposing power lines while maintaining the position of the other of the opposing power lines. In this manner, the distal end of the guidewire is tilted, and the entire guidewire is bent, in the direction of the pulled power line. The power lines are used to power the image sensor, transmit data from the image sensor to an image processor and display and to steer the micro-camera guided guidewire through the body cavity. The ability to power the image sensor and steer the image sensor with the same component, reduces the amount of components required to construct and manipulate the guidewire within the body of the patient. At the micro-scale level of production and use, the ability to minimize components reduces the size of the device.

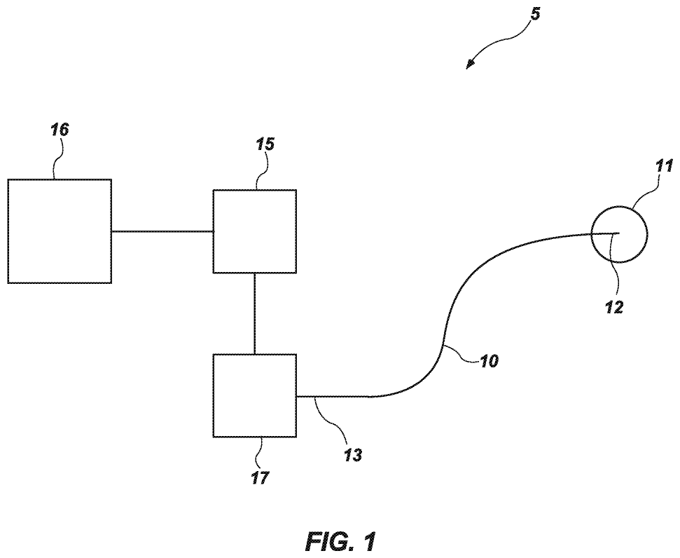

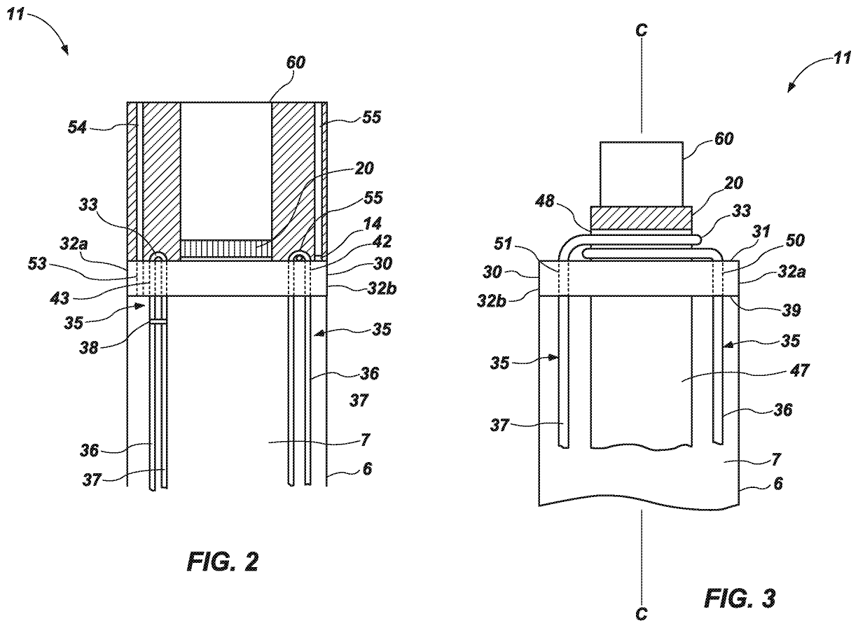

In accordance with one aspect of the technology, with reference to FIGS. 1 and 2, medical imaging system 5 comprises a micro-guidewire 10 having an imaging device that is approximately 700 μm in diameter, shown generally at 11, disposed at a distal tip 12 of the micro-guidewire 10. A processor 15, such as an appropriately programmed computer, is provided to control the imaging system 5 and create an image of the anatomy adjacent the distal tip portion 12 within a patient, displayable on a monitor 16 and storable in data storage as is known in the art. An interface 17 is provided which supplies power to the imaging device 11 and feeds a digital image signal to the processor 15 based on a signal received from the imaging device 11 via conductive wires through the micro-guidewire 10. In accordance with one aspect of the technology, a guidewire 10 is disclosed and is configured for placement into a cavity of a body. The guidewire 10 comprises an elongate body member having a proximal end 13 and a distal end 12. The elongate body member also comprises a light source 14 (e.g., an LED, fiber optic, etc.) and an image sensor 20 disposed about the distal end 12 of the elongate body. In one aspect, the image sensor 20 comprises a solid state imaging device (e.g., a CCD or CMOS device, etc.) with an imaging array 21 disposed thereon. A guide member 30 is disposed about the distal end 12 of the elongate body and the image sensor 20 is disposed about a top surface 31 of the guide member 30. A lens 60 is disposed about a top portion of the image sensor 20 and aligned with the imaging array 21. While specific reference is made to an image sensor 20 comprising a solid state imaging device, it is understood and any number of imaging devices may be used herein as suits a particular purpose. In one aspect of the invention, the lens 60 comprises a GRIN lens. However, it is understood that a number or type of lenses may be used herein as suits a particular purpose and as is known in the art.

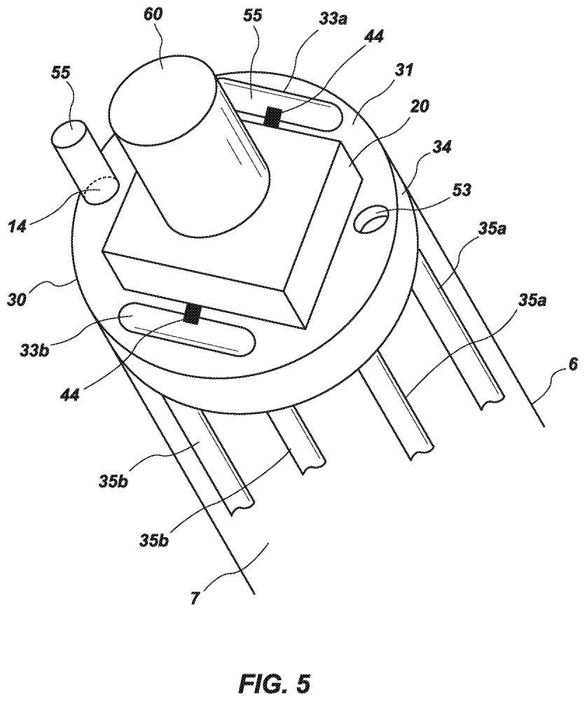

The guide member 30 comprises a plurality of tensioning members 35 disposed about opposing lateral sides 32a, 32b of the guide member 30. The tensioning members 35 are used to steer and manipulate the movement of the distal end 12 of the guidewire 10 by pulling one or more tensioning members 35 while leaving one or more of the tensioning members 35 in position. In this manner, a side (32a or 32b) of the guide member 30 is tilted (e.g., downward) related to the other side of the guide member 30. In one aspect of the technology, each tensioning member 35 comprises a single cable of tensioning material forming a loop (shown generally at 33) about the guide member 30. In one non-limiting example, the tensioning member 35 comprises a conductive material such as copper or aluminum covered with an insulating material. The guide member 30, which houses and is electrically coupled to the image sensor 20, is electrically coupled to the conductive tensioning member 35 such that the tensioning member 35 acts both as a device for delivering power to the image sensor 20 (and the light source 14) and communicating data from the image sensor 20 to the processor 15 and image display device 16. In another aspect of the technology, the tensioning members 35 comprise a cable (or cables) of thermoplastic polymer such as ultra-high-molecular-weight polyethylene (UHMWPE) such as SPECTRA® fibers having yield strengths as high as 2.4 GPa (350,000 psi) and a specific gravity as low as 0.97. In certain aspects of the technology, conductive materials and polymer materials may be used for different individual tensioning members 35 on the same device as suits a particular application and design. For example, with respect to FIG. 5, tensioning member 35a forming loop 33a may comprise a conductive material while tensioning members 35b forming loop 33b may comprise a polymeric material.

Individual tensioning members 35 form a loop 33 about the guide member 30 resulting in two portions 36, 37 of the tensioning members 35 (e.g., which can be referred to as handles) that can be used to steer the guidewire 10. In this manner, the loop 33 is formed from a single cable of material disposed about the guide member 30. Advantageously, the single cable construction minimizes concerns associated with dislodging tensioning members 35 that are soldered to the guidewire 10 or otherwise connected to the guidewire 10 through other means. The single cable construction also simplifies construction. In one aspect of the technology, the two handles 36, 37 resulting from the single loop 33 can be secured together by a coupling member 38 to form a single composite handle used fur steering the distal end 12 of the guidewire 10 and/or powering the image sensor 20. The phrase “single cable” as used herein may comprise a plurality of wires in a winding as is known in the electrical arts to form a single functional cable or a plurality of polymer cables spun or joined together to form a single functional cord or cable.

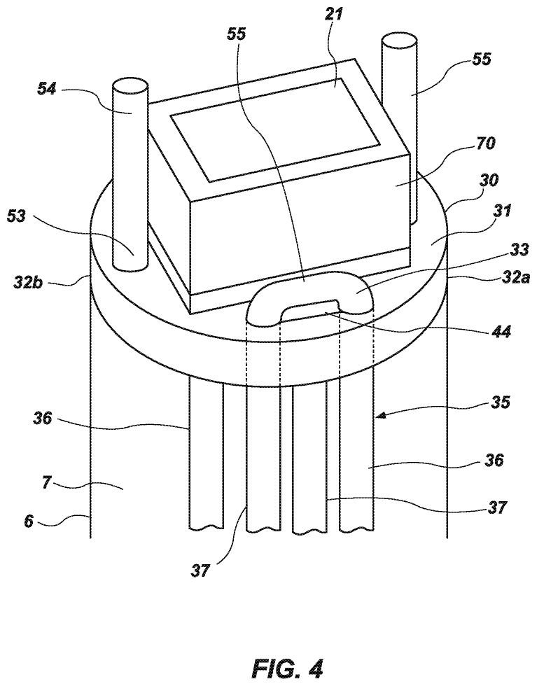

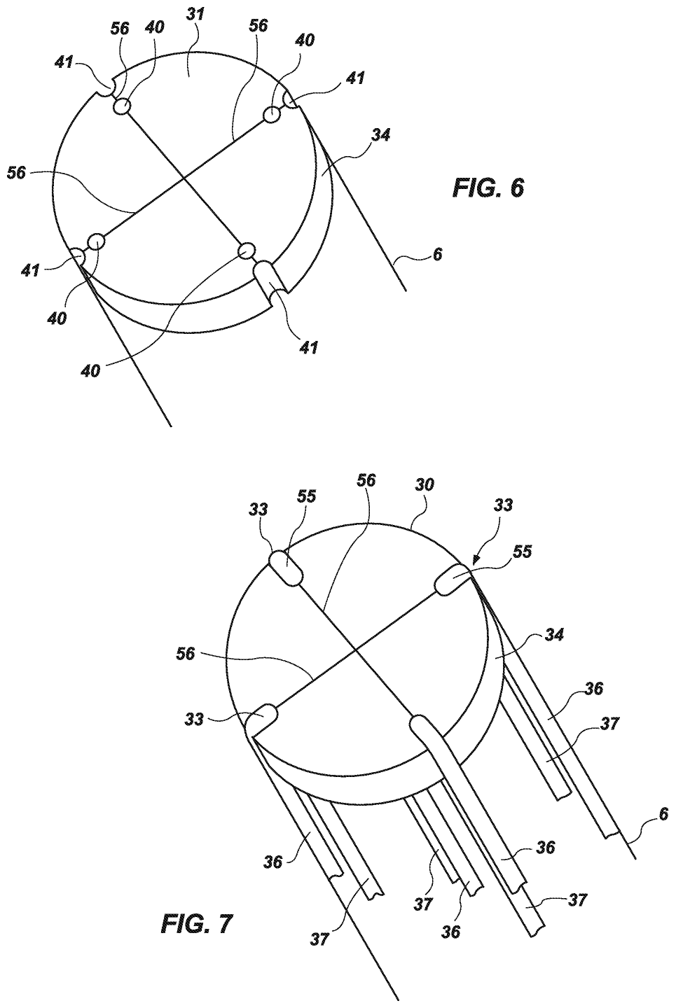

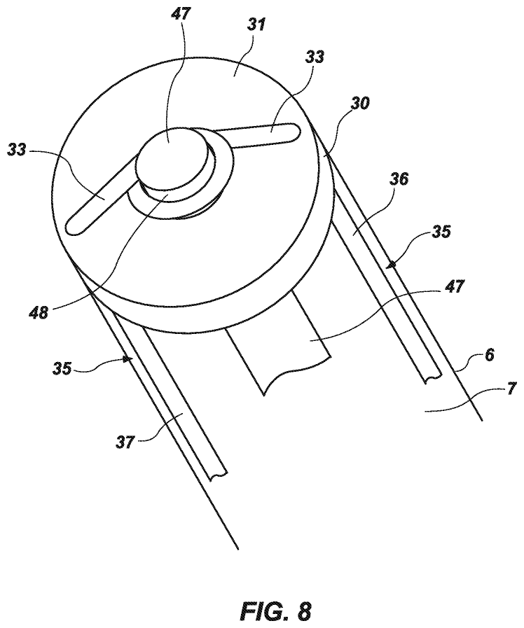

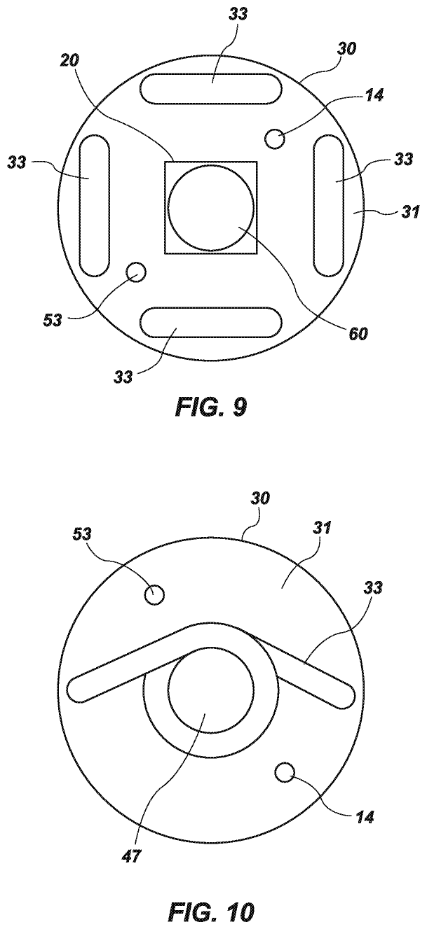

Illustrated in FIGS. 2-4, in one aspect, the loop 33 is formed by inserting a single cable from below the guide member 30, up through an aperture within the guide member 30 and back below the bottom 39 of the guide member 30. This can be accomplished by inserting the cable through an aperture that begins on the bottom 39 of the guide member 30, traverses a portion of the guide member 30 (internally or externally) and exits at a bottom side 31 of the guide member 30. The handles 36, 37 are disposed substantially parallel and adjacent to one another, though they are spaced apart in such a manner as to create a point where the loop 33 engages a surface of the guide member 30 (e.g., at an apex 55) sufficiently to allow the loop 33 of the tensioning member 35 to manipulate movement of the guide member 30 when a force is applied to the tensioning member 35. As shown in FIGS. 6 and 7, in another aspect, the loop 33 is created by inserting a single cable from below the guide member 30, up through an aperture within the guide member 30, across a top surface 31 of the guide member 30, over a side surface 34 of the guide member 30 and back below the guide member 30. The side surface 34 may have a channel 41 configured to receive a portion of the cable therein. In yet another aspect, the loop 33 is created by inserting the single cable member from the below the guide member 30 up through a first aperture 42 within the guide member 30, traversing a portion of a top surface 31 of the guide member 30, and down through a second aperture 43 located in the guide member 30. The distance between adjacent apertures 42, 43 is adjusted based on the size of the apex 55 of the loop 33 as suits a particular application. In one aspect, a plurality of loops 33 EW formed about the guide member 30. In one aspect, four loops 33 are formed about opposing sides of the guide member 30 as is shown in FIG. 9 to facilitate steering and/or powering of the device. However, less than four loops may be used as suits a particular application. For example, the single loop 33 aspect shown in FIGS. 3 and 8 may be used.

In the aspect where the tensioning members 35 comprise a conductive material, the tensioning members 35 are secured (through soldering, adhesives, mechanical clip, or otherwise) to a portion of the guide member 30 that is electrically coupled to the image sensor 20. For example, a soldering joint 44 may be formed on the top 31 of the guide member 30 at the top of the loop 33 formed on the guide member 30. In the aspect where the tensioning members 35 are polymeric materials, they are likewise secured (through adhesives, heat treatment, mechanical clip, or otherwise) to the guide member 30. In accordance with one aspect, and with specific reference to FIG. 6, apertures 40 are electrically connected to one another by way of a conductive material 56 (i.e., copper, aluminum, etc.) that is embedded, deposited, or otherwise disposed on the guide member 30. In one aspect, the conductive material 56 traverses a top surface 31 of the guide member 30 and is in contact with the image sensor 20. A conductive material 56 may also be disposed within a portion of the aperture 40 and/or channel 41 to maximize contact between electrically conductive tensioning members, the conductive material 56 located within the apertures 40 in which the tensioning members 35 are disposed, and an electrical coupling with the image sensor 20.

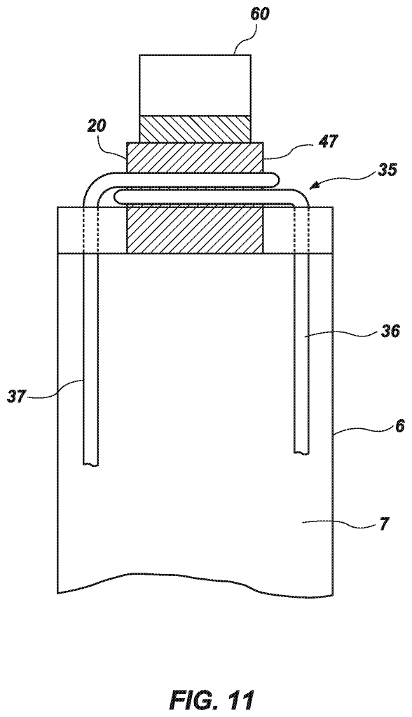

With reference now to FIGS. 3, 8, and 10, in one aspect of the technology, a single loop 33 is created by inserting a tensioning member 35 up through a first aperture 50 in the guide member 30, substantially traversing the diameter (where the guide member 30 is circular, a length if it is rectangular, etc.) of the guide member 30 and exiting the bottom of the guide member 30 on an opposing side of the guide member 30. In one aspect, the tensioning member 35 may traverse an internal portion of the guide member 30 or, as is shown in the drawings, may traverse a top surface 31 of the guide member 30 and down through a second aperture 51 back below the guide member 30. In any event, a pair of handles 36, 37 is created by the opposing sides of the loop 33. In this aspect, the single cable traverses a substantial portion of the guide member 30 so as to enable a pivoting action creating by pulling one of the handles 36, 37 of the loop 33. In one aspect of the technology, a core member 47 is disposed about the center of the guide member 30. The core member 47 may comprise a semi-rigid material (e.g., Nitinol, stainless steel, titanium, etc.) used to provide rigidity to the guidewire during advancement of the guidewire within the vasculature (or other body cavity) of the patient while being pliable enough to navigate tortuous paths found within the body with minimal damage to the vasculature of the patient. In one aspect of the technology, the single cable is coupled to the core member 47. The single cable is disposed about an exterior 48 of the core 47, circumscribing the core 47. In one aspect, the cable is disposed through an aperture of the core 47 and secured (by adhesive, bonding, or otherwise) to the core 47. Advantageously, as forces are exerted on either one or both of the handles 36, 37 to manipulate the guide member 30, the core 47 is also moved in tandem with the guide member 30. In one aspect of the technology, an image sensor 20 and complementary lens 60 are disposed about the core 47 and are collinear with the core 47. In this manner, as the guide member 30 is manipulated, the image sensor 20 and lens 60 are likewise more precisely moved in tandem with the guide member 30. In this aspect, where it is desired to use conductive materials as the tensioning members 35 disposed about the core 47, the tensioning members 35 and/or the core 47 are electrically coupled to the image sensor 20 and the tensioning members 35 are secured (adhered, bonded, soldered, or otherwise) to the core 47. While the figures show the core 47 protruding from a top surface 31 of the guide member 30, it is understood that the core 47 need not have such a construction. The core 47 could terminate at the top surface 31 of the guide member 30 or within the guide member 30, the cable of the tensioning member 35 traversing an internal portion of the guide member 30. In another aspect of the technology, shown in FIG. 11, the core 47 is located within the guide member only and does not extend through the hollow elongate body 6 of the guidewire. In this manner, a smaller internal diameter of the hollow elongate body 6 is achieved as only a single tensioning member 35 is used to power and steer the device.

In one aspect of the technology, the guidewire 10 comprises a fluid-tight hollow elongate body 6. The elongate body 6 may be made from a semi-rigid polymeric material such as a polyurethane, polyamide, fluoropolymer, polyolefins, or other polymers known in the art. The tensioning members 35 extend downward from the guide member 30 on the distal end 12 of the guidewire 10 to the proximal end 13 of the guidewire 10 through the hollow elongate body 6. The hollow elongate body 6 serves to house the tensioning members 35. In an aspect where the tensioning members 35 are sterile and the internal cavity 7 of the hollow elongate body 6 is sterile, the hollow body 6 may serve to communicate a fluid to a distal end 12 of the guidewire 10. The apertures 40, 42, 43, 50, 51, etc. receiving the tensioning members 35 are sealed to prevent fluid transfer into or from the hollow body 6 of the guidewire 10. However, a separate aperture 53 is disposed within the guide member 30 having a valve disposed therein. The valve is configured to permit the injection of fluids (e.g., a contrasting agent or other imaging fluid) from the hollow body 6 of the guidewire 10 and aspirate fluid into the hollow body 6 of the guidewire 10. A non-limiting example of a valve capable of use in this aspect includes a two-way slit valve or other two-way valves known in the art.

In one aspect of the technology, the lens 60 is formed with a substantially sealed distal end 12 of the guidewire 10. The end may be rectangular or may have rounded edges to facilitate movement within the vasculature of the patient. An elongate member 54 is formed or disposed in the sealed distal end 12 to communicate with aperture 43 in order to aspirate and inject fluids and an elongate member 55 is disposed or formed in the sealed distal end 12 to communicate light from light source 14. In another aspect of the technology, the distal end 12 of the guidewire 10 is completely sealed such that there is no fluid passage to or from the interior of the guidewire 10. In one aspect, the lens 60, image sensor 20, and guide member 30 are encapsulated in an opaque epoxy matrix to seal the distal end 12 of the guidewire 10.

As the guidewire 10 is navigated through the body, an increased view of a target is created by tiling images from the imaging sensor 20 from a plurality of adjacent image capture areas. The adjacent image capture areas are tiled together over time to create a longitudinally continuous view of the body cavity. By way of example, and without limitation, an initial 360-degree view may be thought of as creating an annular image by stitching together a plurality of views as the camera is moved in a circular pattern.

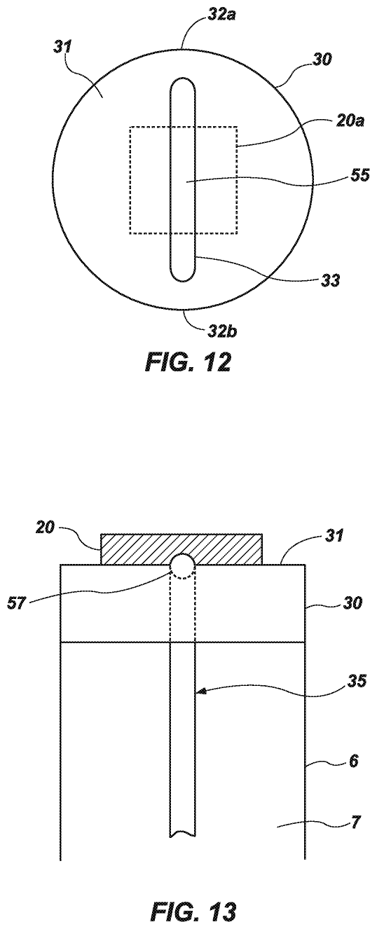

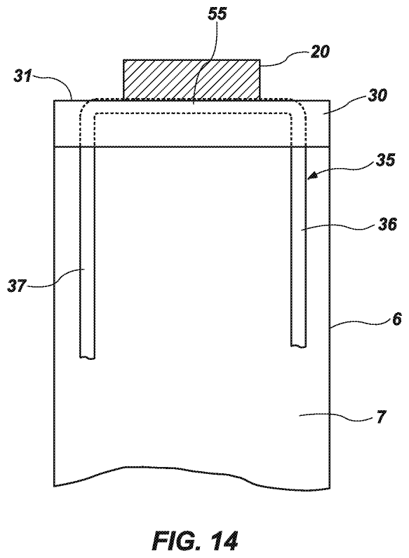

With reference to FIGS. 12 and 13 generally, in accordance with one aspect of the technology, a channel 57 is disposed about a top surface 31 of the guide member 30. The tensioning member 35 is disposed in the channel 57. The depth of the channel 57 is configured such that a bottom portion of the image sensor 20, once placed on the top surface 31 of the guide member 30, is in contact with a bottom surface of the image sensor 20. Footprint 20a shows an example location where an imaging sensor 20 can be placed on the guide member 30 and would be in electrical connection with the imaging sensor 20. Similar to aspects of the technology described herein, a single cable is used to create a loop 33 about the guide member 30 that functions as a tensioning member 35 and may also function as a power line for the image sensor 20.

Methods of using the devices described herein are apparent to one of ordinary skill in the art. In a non-limiting example, a method of placing a guidewire into a cavity of a body is described. Guidewires are small elongate objects used to navigate paths of the body to a specific target area of the body requiring treatment. In lieu of performing open surgery on a patient, a guidewire is placed into the body until it reaches the target area. Once the target area is reached, a catheter (a hollow tube) is placed over the guidewire. Once the catheter is placed into the body cavity over the same path as the guidewire, the guidewire is removed. In one aspect, the method comprises placing a guidewire in the body of the patient. An image sensor is disposed about a guide member on a distal end of the guidewire and a plurality of power lines are operatively coupled to the guide member and the image sensor. The at least two power lines of the plurality of power lines are disposed on opposing sides of the guide member and extend from the distal end of the guidewire to a proximal end of the guidewire. The guidewire is advanced through the body cavity while viewing the internal area of the body cavity with a display device coupled to the image sensor. The distal end of the guidewire is positioned within the body cavity by applying a force (e.g., pushing or pulling) on one of the at least two power lines (which function as tensioning members) while maintaining the position of the other of the at least two power lines. Once a target location within the body cavity is reached, a catheter is placed about an exterior of the guidewire and advanced through the body cavity. In one aspect, the guidewire is then removed from the body cavity while leaving the catheter in the body cavity, though in other aspects the guidewire remains in place as multiple catheters are used during a single procedure. Advantageously, there is no separate viewing device (external or internal) required for viewing the advancement and/or final placement of the guidewire within the cavity of the body. In another aspect, a single power line is disposed about the guide member and configured such that the single power line is coupled to the guide member about opposing sides of the guide member forming a loop about the guide member with opposing handles being formed from the single power line. Force is applied to the opposing handles in order to manipulate movement of the guide member and hence the guidewire through the vasculature (or other body cavity) of the patient.

The foregoing detailed description describes the technology with reference to specific exemplary aspects. However, it will be appreciated that various modifications and changes can be made without departing from the scope of the present technology as set forth in the appended claims. The detailed description and accompanying drawings are to be regarded as merely illustrative, rather than as restrictive, and all such modifications or changes, if any, are intended to fall within the scope of the present technology as described and set forth herein.

More specifically, while illustrative exemplary aspects of the technology have been described herein, the present technology is not limited to these aspects, but includes any and all aspects having modifications, omissions, combinations (e.g., of aspects across various aspects), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus-function are expressly recited in the description herein. Accordingly, the scope of the technology should be determined solely by the appended claims and their legal equivalents, rather than by the descriptions and examples given above.

1. A method of advancing a guidewire having an imaging device disposed thereon within a cavity of a body, comprising:
placing a guidewire into a cavity of a body, said guidewire comprising an image sensor disposed about a guide member on a distal end of the guidewire and a plurality of power lines operatively coupled to the guide member and the image sensor, wherein at least two power lines of the plurality of power lines are disposed on opposing sides of the guide member and extend from the distal end of the guidewire to a proximal end of the guidewire;
advancing the guidewire through the body cavity while viewing an internal area of the body cavity with a display device coupled to the image sensor to a target location within the body;
positioning the distal end of the guidewire within the body cavity applying a force to one of the at least two power lines while maintaining the position of the other of the at least two power lines;
placing a catheter about an exterior of the guidewire and advancing the catheter through the body cavity;
removing the guidewire from the body cavity while leaving the catheter in the body cavity.

2. The method of claim 1, wherein the guide member comprises a plurality of apertures disposed within the guide member, the apertures passing from a top surface of the guide member to a bottom surface of the guide member.

3. The method of claim 2, wherein each of the at least two power lines comprise a single cable of conductive material forming a loop through the guide member.

4. The method of claim 3, wherein the cable of each power line extends from below the guide member, up through a first aperture within the guide member, over the top surface of the guide member, and down through a second aperture within the guide member to below the guide member.

5. The method of claim 4, wherein the guidewire comprises a fluid-tight hollow elongate body, the power lines extending from the guide member on the distal end of the guidewire to the proximal end of the guidewire.

6. The method of claim 5, wherein the first and second apertures associated with the power lines are sealed to prevent fluid from passing from the fluid-tight hollow elongate body through the first and second apertures.

7. The method of claim 6, wherein a pressurized fluid is communicated from the fluid-tight hollow elongate body through a third aperture disposed within the guide member into the body cavity.

8. A method of advancing a guidewire having an imaging device disposed thereon within a cavity of a body, comprising:
placing a guidewire into a cavity of a body, said guidewire comprising (i) an image sensor disposed about a guide member on a distal end of the guidewire, and (ii) a single power line operatively coupled to the guide member and the image sensor, wherein the single power line extends from a bottom of the guide member up through a first aperture in the guide member across the guide member and down through a second aperture in the guide member, the first and second apertures being disposed on laterally opposing sides of the guide member, creating opposing cables;
advancing the guidewire through the body cavity; and
positioning the distal end of the guidewire within the body cavity by pulling on one of the opposing cables of the single power line while maintaining the position of the opposing cable of the single power line.

9. The method of claim 8, wherein the guidewire further comprises a semi-rigid core disposed about a center of the guide member.

10. The method of claim 9, wherein single the power line is coupled to the semi-rigid core.

11. The method of claim 9, wherein the single power line extends from the first aperture, circumscribing an outer perimeter of the core, and into the second aperture.

12. The method of claim 9, adjusting the lateral position of the distal end of the guidewire within the body cavity by moving a distal end of the core in a lateral direction.

13. A guidewire configured for placement into a cavity of a body, comprising:
an elongate body member having a proximal end and a distal end, the elongate body member comprising a light source and an image sensor disposed about the distal end of the body;
a guide member disposed about the distal end of the elongate body, wherein the image sensor is disposed about a top surface of the guide member;
a plurality of tensioning members disposed about opposing lateral sides of the guide member, wherein each tensioning member comprises a single cable of tensioning material forming a loop about the guide member, said loop extending from below the guide member, up through an aperture within the guide member and back below a bottom of the guide member.

14. The guidewire of claim 13, wherein the loop extends from below the guide member, up through the aperture within the guide member, across a top surface of the guide member, and back below the guide member.

15. The guidewire of claim 13, wherein the loop extends from below the guide member, up through the aperture within the guide member, over a side surface of the guide member, and back below the guide member.

16. The guidewire of claim 13, wherein the loop extends from below the guide member, up through a first aperture within the guide member, across a top surface of the guide member, through a second aperture within the guide member, and back below the guide member.

17. The guidewire of claim 13, wherein the tensioning members comprise a conductive material configured to communicate power to the image sensor.

18. The guidewire of claim 13, wherein the tensioning members comprise UHMWPE fibers.

19. The guidewire of claim 13, wherein the guidewire comprises a fluid-tight hollow elongate body, the tensioning members extending from the guide member on the distal end of the guidewire to the proximal end of the guidewire through the hollow elongate body.

20. The guidewire of claim 19, wherein the aperture receiving the tensioning members is sealed to prevent fluid transfer into or from the hollow body of the guidewire.

21. The guidewire of claim 20, further comprising the aperture within the guide member having a valve disposed therein, said valve configured to permit the injection of fluids from the hollow body of the guidewire and aspirate fluid into the hollow body of the guidewire.

22. A guidewire configured for placement into a cavity of a body, comprising:
an elongate body member having a proximal end and a distal end, the elongate body member comprising a light source and an image sensor disposed about the distal end of the body;
a guide member disposed about the distal end of the elongate body, wherein the image sensor is disposed about the guide member; and
a single cable tensioning member extending (i) from a bottom of the guide member up through an aperture in the guide member, (ii) laterally across the guide member, and (iii) back below a bottom of the guide member.

23. The guidewire of claim 22, wherein the single tensioning member forms a loop about a top surface of the guide member, the single cable tensioning member having first and second handles extending about opposing sides of the guide member.

24. The guidewire of claim 22, further comprising a semi-rigid core disposed about a center of the guide member.

25. The guidewire of claim 24, wherein the single tensioning member is coupled to the semi-rigid core.

26. The guidewire of claim 24, wherein the single tensioning member is disposed through the semi-rigid core.

27. The guidewire of claim 24, wherein the single tensioning member is disposed about an exterior surface of the semi-rigid core.
